(19) United States
(12) Patent Application Publication
(10) Pub. No.: US 2025/0313541 A1
(43) Pub. Date: Oct. 9, 2025

(54) EPOXIDATION PROCESS WITH IMPROVED ETHYLENE SEPARATION AND RECOVERY

(71) Applicant: Scientific Design Company, Inc., Little Ferry, NJ (US)

(72) Inventor: Saurabh KATIYAR, Little Ferry, NJ (US)

(73) Assignee: Scientific Design Company, Inc., Little Ferry, NJ (US)

(21) Appl. No.: 19/171,693

(22) Filed: Apr. 7, 2025

Related U.S. Application Data
(60) Provisional application No. 63/575,121, filed on Apr. 5, 2024.

(51) Int. Cl. C07D 303/04

(52) U.S. Cl. CPC C07D 303/04

(57) ABSTRACT

A process for the preparation of ethylene oxide in which the efficiency of separating ethylene from carbon dioxide is improved.

### CROSS REFERENCE TO RELATED APPLICATION

The present invention claims the benefit of U.S. Provisional Patent Application No. 63/575,121 filed Apr. 5, 2024, the entire content and disclosure of which is incorporated herein by reference.

### FIELD OF THE INVENTION

The present invention relates to a method for the oxidation of ethylene into ethylene oxide.

### BACKGROUND OF THE INVENTION

Though present in natural settings at minute quantities, ethylene oxide was first synthesized in a laboratory setting in 1859 by French chemist Charles-Adolphe Wurtz using the so-called “chlorohydrin” process. However, the usefulness of ethylene oxide as an industrial chemical was not fully understood in Wurtz's time; and so industrial production of ethylene oxide using the chlorohydrin process did not begin until the eve of the First World War, due at least in part to the rapid increase in demand for ethylene glycol (of which ethylene oxide is an intermediate) as an antifreeze for use in the rapidly growing automobile market. Even then, the chlorohydrin process produced ethylene oxide in relatively small quantities and was highly uneconomical.

The chlorohydrin process was eventually supplanted by another process, the direct catalytic oxidation of ethylene with oxygen, the result of a second breakthrough in ethylene oxide synthesis, discovered in 1931 by another French chemist, Theodore Lefort. Lefort used a solid silver catalyst with a gas phase feed that included ethylene and utilized air as a source of oxygen.

In the ninety years since the development of the direct oxidation method, the production of ethylene oxide has increased so significantly that today it is one of the largest volume products of the chemicals industry, accounting, by some estimates, for as much as half of the total value of organic chemicals produced by heterogeneous oxidation. Worldwide production in the year 2000 was about 25 billion tons. One of the reasons that ethylene oxide is such a widely produced chemical product is its startling versatility—it is the starting point for innumerable derivatives, including ethylene glycol, ethoxylates, ethanolamines, polyols, and glycol ethers, each of which becomes the raw material for numerous high-value products such as fabrics, moldable plastics, surfactants, detergents, solvents and many others.

Increases in annual production of ethylene oxide and ethylene glycol have been further enabled by parallel increases in production plant sizes. While larger plants are more efficient, they nonetheless present certain challenges. For example in a typical ethylene oxide plant, the reactor effluent usually contains not only the desired ethylene oxide product but also products of the incomplete epoxidation reaction, primarily carbon dioxide, as well as unreacted gases, especially ethylene. In essence, the ethylene is “contaminated” with carbon dioxide, which must be removed so that ethylene can be recycled back to the reactor. But in these larger plants, this problem is made more complicated because even greater amounts of byproducts and unreacted gases can make separation and purification more difficult and energy-intensive. Increases in the amount of carbon dioxide made under these more exacting process conditions may require a larger carbon dioxide absorber and more utility import for steam-stripping in the carbon dioxide regenerator. Moreover, the larger volume of the fluid process streams moving through these larger plants can increase capital costs because of the necessity of upsizing of other equipment besides the carbon dioxide absorber to handle these larger streams. Thus, for these larger plants it is especially essential that unreacted feed gases like ethylene need to be recycled back to the reactor to improve process efficiency.

Accordingly, there is a need in the art for a process that efficiently separates carbon dioxide and ethylene and recovers this ethylene for recycling back to the reactor. By operating more efficiently, higher operating costs can be avoided, and furthermore the process configured in such a way as to avoid increasing capital or equipment costs.

### BRIEF SUMMARY OF THE INVENTION

The present invention relates to a process for the preparation of ethylene oxide stream comprising the steps of: providing scrubber overheads comprising about 25 vol % to about 35 vol % ethylene; in a carbon dioxide absorber, contacting the scrubber overheads with a carbon dioxide-absorbing solvent to form a remaining gas stream and a rich carbonate solution, the rich carbonate solution comprising ethylene having a temperature of about 65° C. to about 85° C.; passing the rich carbonate solution under a pressure of between about 10 atm to about 20 atm to and through a first heat exchanger, where the temperature of the rich carbonate solution is raised to an elevated temperature after exiting the first heat exchanger; directing the rich carbonate solution from the first heat exchanger to and through a second heat exchanger, where the temperature of the rich carbonate solution is raised to about 5° C. to about 20° C. above the elevated temperature; dividing, in a first flash drum, the rich carbonate solution into a first flash drum overhead and a first flash liquid; separating, in a second flash drum, a second flash liquid from the first flash liquid; passing the second flash liquid to a regenerator; and dividing, in the regenerator, the second flash liquid into a lean solvent and a gaseous regenerator overhead steam comprising carbon dioxide and ethylene, wherein the amount of ethylene is less than about 50 ppm, preferably less than about 20 ppm (on weight basis).

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a schematic flow sheet showing a process for making ethylene oxide according to the present invention.

FIG. 2 is a detail of FIG. 1 showing the construction and operation of the flash drum regenerator 25 as an integrated process column.

### DETAILED DESCRIPTION OF THE INVENTION

All parts, percentages and ratios used herein are expressed by volume unless otherwise specified. All documents cited herein are incorporated by reference.

By the present invention, an improved ethylene recovery scheme has been incorporated into the manufacture of ethylene oxide to increase the amount of ethylene that is recovered and recycled back to the reactor. This both decreases the operating cost of ethylene oxide manufacture and because there is far less waste ethylene for remediation, increases the efficiency of the process.

The present invention makes use of two heat exchangers through which the rich carbonate solution from the bottoms of the carbon dioxide absorber 15 flows sequentially. Heating the rich carbonate solution to a high temperature greatly improves the efficiency of the separation of the ethylene in the rich carbonate solution when the solution is supplied to the flash drums contained in the flash drum regenerator.

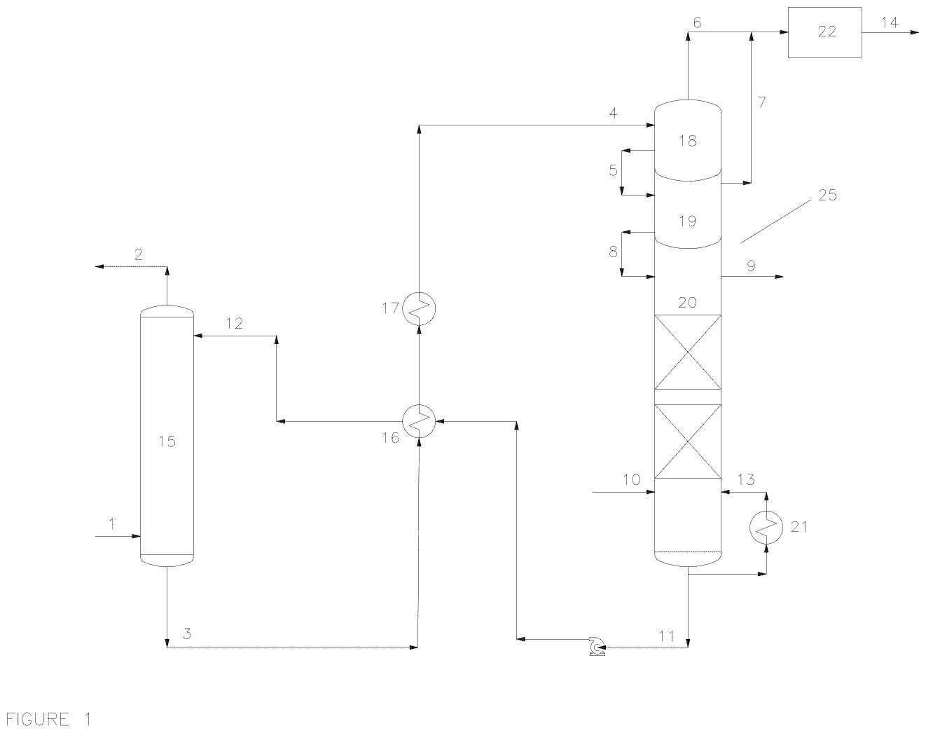

This invention will now be described in greater detail as a component of an ethylene oxide production process. The invention itself is shown with specificity in FIG. 1.

Ethylene oxide is produced by continuously contacting an oxygen-containing gas with an olefin, preferably ethylene, in the presence of an ethylene oxide (“epoxidation”) catalyst (described in greater detail below). Oxygen may be supplied to the reaction in substantially pure molecular form or in a mixture such as air. By way of example, typical reactant feed mixtures (after the completion of start-up and during normal operation) typically comprise from about 0.5% to about 45%, preferably about 5% to about 30% of ethylene and from about 3% to about 15% oxygen, and from about 0.3% to about 10%, preferably from about 0.1% to about 1%, carbon dioxide with the balance comprising comparatively inert materials, including such substances as water, inert gases, other hydrocarbons, and the reaction moderators described herein. Inert hydrocarbons include, but are not limited to, methane and ethane; the reactant feed mixtures may contain from about 0.5% to about 45% methane and up to 3% ethane. Non-limiting examples of inert gases include nitrogen, argon, helium and mixtures thereof. Non-limiting examples of the other hydrocarbons include methane, ethane, propane and mixtures thereof. Carbon dioxide and water are byproducts of the epoxidation process as well as common contaminants in the feed gases. Both have adverse effects on the catalyst, so the concentrations of these components are usually kept at a minimum. (All percentage compositions listed in the present paragraph are in volume %.)

Also present in the reaction, as previously mentioned, are one or more reaction moderators, non-limiting examples of which include organic halogen-containing compounds such as C₁ to C₈ halohydrocarbons; especially preferred are chloride-containing moderators such as methyl chloride, ethyl chloride, ethylene dichloride, vinyl chloride or mixtures thereof. Controlling chloride concentration level is particularly important with rhenium-containing catalysts.

As mentioned above, a usual method for the ethylene epoxidation process comprises the vapor-phase oxidation of ethylene with molecular oxygen, in the presence of a silver-based epoxidation catalyst, in a fixed-bed tubular reactor. Conventional, commercial fixed-bed ethylene-oxide reactors are typically in the form of a plurality of parallel elongated tubes (in a suitable shell) approximately 0.7 to 2.7 inches O.D. and 0.5 to 2.5 inches I.D. and 15-53 feet long, each filled and packed with catalyst. The reaction feed mixture (described above) is introduced into these tubes, and the resulting reactor effluent gas contains ethylene oxide, un-used reactants, and byproducts. Typically in the ethylene oxide process, the typical work rate for the reactor is between 130 and 300 kg/m³/h, while the ΔEO is between 1.0% and 2.5%. The work rate is the production rate and is represented herein by the units kg/m³/h. The ΔEO is defined as the moles of EO formed in the reactor per 100 moles of reactor feed and essentially represents the concentration of ethylene oxide in the reactor effluent, since the concentration of ethylene oxide in reactor feed must be maintained at very close to zero, indeed typically only a few ppm.

Typical operating temperatures for the reactor (as measured in the shell side coolant of the reactor) in the range from about 180° C. to about 330° C., and preferably from about 200° C. to about 325° C., and more preferably from about 225° C. to about 280° C. The operating pressure may vary from about 1 atmosphere to about 30 atmospheres, depending on the mass velocity and productivity desired. Higher pressures may be employed within the scope of the invention. Residence times in commercial-scale reactors are generally on the order of about 2 to about 20 seconds.

In operation, reactor effluent from the reactor outlet (not shown) flows to a scrubber (not shown) for the recovery of the ethylene oxide product. The reactor effluent contains from about 1.0 mol % to about 2.5 mol % ethylene oxide, preferably about 1.8 mol % to about 2.2. mol %.

In addition to ethylene oxide, the reactor effluent may also contain inert and unreacted gases supplied as components of the reactor feed such as argon, methane, ethylene and oxygen; reaction byproducts, in particular, carbon dioxide, but also ppm levels of formaldehyde, formic acid, acetic acid, and product isomers such as acetaldehyde; and additionally sulfur and other impurities, again typically at ppm or ppb levels. The byproducts and impurities intermixed with ethylene oxide can undermine the quality of downstream products such as ethylene glycol. Feedstock materials such as methane, ethylene and oxygen must be recovered and recycled back to the reactor inlet feed in order increase the economic efficiency of the ethylene oxide process, and because if not recovered, for environmental reasons the hydrocarbons can be vented at only the most minute quantities.

The scrubber is the first step in the separation and recovery process and is where most of the ethylene oxide and light gases are removed from the reactor effluent. In the scrubber, the reactor effluent is contacted with lean cycle water. The temperature of the lean cycle water and pressure in the scrubber is managed to maximize the amount of ethylene oxide absorbed into the water and minimize the absorption of other components, especially carbon dioxide. This takes advantage of the very different degrees to which ethylene oxide and carbon dioxide are soluble in water: for example, at ambient temperature, ethylene oxide is essentially infinitely soluble in water; while carbon dioxide is only moderately soluble. Those gases that are not absorbed in the cycle water, but rise upward to the top of the scrubber to from the scrubber overheads. The gases in the scrubber overhead are not just carbon dioxide, but also the aforementioned feed gases such as methane, ethylene and oxygen. While gases such as methane have even lower solubility in water than carbon dioxide, they are present in much greater quantities in the reactor effluent than carbon dioxide and so are present in much higher quantities in the scrubber overhead.

Specifically, the scrubber overhead comprises about 30 vol % to about 45 vol % methane and 25 vol % to about 35 vol % ethylene (which has passed through the reactor without reacting with oxygen); and about 0.5 vol % to 5 vol % carbon dioxide. As mentioned above, in essence these valuable hydrocarbons in the reactor overhead are “contaminated” with carbon dioxide, which must be removed so that the ethylene and methane in the scrubber overhead stream can be recycled back to the reactor. Given this high amount of ethylene seen in the scrubber overheads, it would be economically disastrous (not only regulatorily impermissible) to simply vent this stream. Rather, the ethylene must be recovered and recycled back to the reactor. It should be noted for the purpose of this invention the amounts of other reactor feed components, such as oxygen, and other hydrocarbons, such as methane, are not taken into account and the description of the invention will proceed solely with respect to ethylene.

As shown in FIG. 1, these scrubber overheads are then supplied to the carbon dioxide absorber 15. The carbon dioxide absorber 15 has the important role of preferentially removing carbon dioxide from the scrubber overheads 1 to allow recycle of the scrubber overheads to the reactor, and thus, the recovery of ethylene contained in the scrubber overheads. Thus, the carbon dioxide absorber 15 is operated at a temperature and pressure to minimize the absorption of hydrocarbons into the carbon dioxide-absorbing solvent.

The carbon dioxide absorber 15 makes use of conventional equipment and techniques known to the person of ordinary skill for removing carbon dioxide from a gaseous feed. In operation, the (carbon dioxide-containing) scrubber overhead stream 1 is contacted with a carbon dioxide-absorbing solvent so that carbon dioxide is preferentially and readily solubilized and absorbed into the solvent. Specifically, as shown in FIG. 1, the (carbon dioxide-containing) scrubber overhead 1 is contacted with a carbon dioxide-absorbing solvent (which has been recycled from the regenerator section 20) in the carbon dioxide absorber 15 to form a carbon dioxide-rich solvent phase and a carbon dioxide-depleted gas phase. The carbon dioxide-absorbing solvent preferentially absorbs carbon dioxide so that the hydrocarbons and oxygen in the scrubber overheads 1 are not absorbed and these form the carbon dioxide-depleted gas phase in the carbon dioxide absorber 15 overhead. Preferably, the carbon dioxide-absorbing solvent is potassium carbonate; the carbon dioxide reacts with the potassium carbonate to form potassium bicarbonate thereby removing the carbon dioxide and forming a potassium bicarbonate-rich solvent phase.

In a particularly preferred embodiment the steps in the carbon dioxide absorber are carried out by reactive distillation.

It should be noted that FIG. 1 shows 100 vol % of the scrubber overheads 1 being sent to the carbon dioxide absorber 15 for carbon dioxide removal and none is bypassed around the carbon dioxide absorber and recycled back to the reactor inlet without treatment in the carbon dioxide absorber to remove carbon dioxide. Regardless of the exact percentage of scrubber overheads 1 that is sent to the carbon dioxide absorber 15, it must be sufficiently high in order to reduce the amount of carbon dioxide to relatively low levels—as mentioned above carbon dioxide is a byproduct of the epoxidation process and it has an adverse effect on the performance of high selectivity catalysts, and so the total amount of carbon dioxide in the streams recycled back to the reactor should be minimized. For lower carbon dioxide concentrations, for example to maintain the 1 vol % carbon dioxide in the inlet feed to a reactor containing high selectivity catalyst, close to 100% of the scrubber overheads 1 must be fed to the carbon dioxide absorber 15. Any stream that “bypasses” the carbon dioxide absorber 15 is preferably recycled back to the reactor inlet because while it would be possible to simply vent the untreated bypass stream to the atmosphere, this would almost never be done intentionally given the loss of valuable hydrocarbons in the bypass stream and the requirement for extensive emissions treatment prior to venting. Nonetheless, it is a possible under the operational constraints of emergencies that the bypass stream could be partially vented.

At the bottom of the carbon dioxide absorber 15 the carbon dioxide-rich solvent phase exits the carbon dioxide absorber 15 as the rich carbonate solution 3. The rich carbonate solution 3 is rich in carbon dioxide stored in the form of a compound like the potassium carbonate described above. The temperature of the rich carbonate solution 3 is about 65° C. to about 85° C. While the absorber is operated in the present invention to preferentially absorb only carbon dioxide and minimize the absorption of other gases, some hydrocarbons, in particular ethylene, have a minimal amount of solubility in the carbon dioxide-absorbing solvent. Accordingly, this rich carbonate solution 3 contains from about 0.01 weight (i.e., wt) % to about 0.5 wt % of ethylene. As discussed above, there are only two options for this ethylene, either it is recovered and recycled back to the reactor or it is vented to the atmosphere. Given that venting is not only impractical due to environmental regulations, but also is an economically wasteful treatment of valuable feedstock, it is necessary to recover even the small amount of ethylene and possibly other hydrocarbons absorbed in the rich carbonate solution.

In the present invention, the amount of ethylene and other hydrocarbons that is recovered from the rich carbonate solution 3 is increased significantly compared to previous ethylene oxide processes so that substantially all the ethylene absorbed in the rich carbonate solution 3 is recovered. By “substantially all” it is meant that the regenerator overhead 9 contains less than 20 ppm (on molar basis) of ethylene. This means that from the ethylene in the rich carbonate solution, which contains up to 0.5 wt % ethylene, there will be less than 50 ppmw, preferably less than 20 ppmw of that ethylene remains unrecovered in the regenerator overhead after being flashed in the first and second flash drums 18, 19 and sent to the regenerator 20 (this is discussed in greater detail below). At these minute levels, the ethylene may be safely vented to the atmosphere and such small losses of ethylene are not noticeable in the overall economics of the plant.

To recover these large amounts of ethylene in process, the present invention makes use of two sequential heat exchangers shown in FIG. 1 as 16 and 17. These heat exchangers heat the rich carbonate solution 3 to temperatures which increase the release of dissolved ethylene when flashed, while also economically making use of available sensible heat from other streams. In addition to the increase in the amount of ethylene oxide that is recovered, using two exchangers 16 and 17 also reduces cost. Because the rich carbonate solution 3 is under such high pressures (as mentioned above the pressure in the carbon dioxide absorber can be as higher as 20 atm), the cost of fabricating a single heat exchanger capable of effecting the desired temperature increase of the rich carbonate solution becomes so prohibitive that it is more economical to use two smaller heat exchangers capable of withstanding this pressure rather than a single larger heat exchanger.

From the carbon dioxide absorber 15, the rich carbonate solution 3 flows or passes under a pressure between about 10 to 20 atm (absolute) through the first of these exchangers, i.e., first heat exchanger 16, and exchanges heat with the regenerator bottoms 11 coming from regenerator section 20 of the flash drum regenerator 25 (the operation of the regenerator is described in detail, below). The rich carbonate solution 3 is thus heated by indirect heat exchange with the regenerator bottoms 11 so that after passing through the first heat exchanger 16 the temperature of the rich carbonate solution 3 is raised from a temperature of about 65° C. to about 85° C. prior to entering first heat exchanger 16 to an elevated temperature, the elevated temperature being about 95° C. to about 110° C. after exiting the first exchanger outlet of first heat exchanger 16. This heating is accomplished solely through the recovery of the sensible heat from the regenerator bottoms 11, without the necessity of any additional energy input.

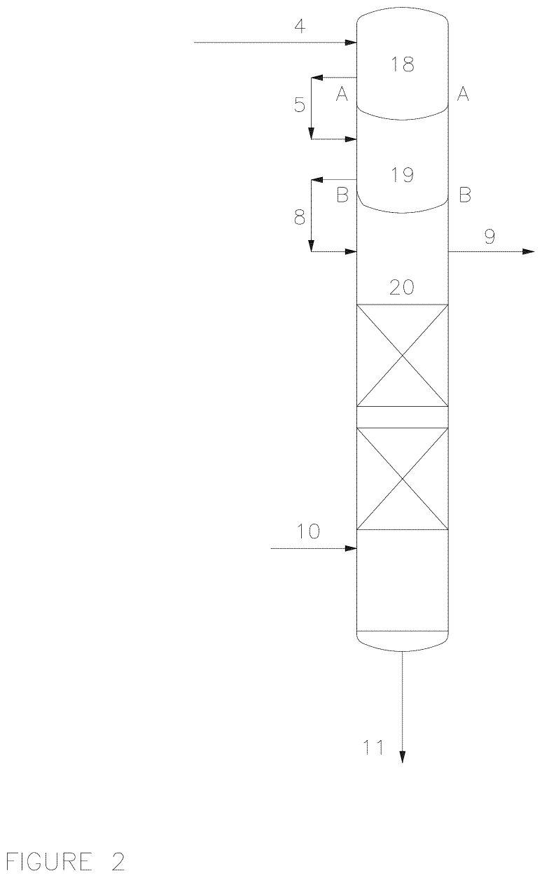

FIGS. 1 and 2 show the preferred embodiment of the present invention where the first flash drum 18, second flash drum 19, and regenerator 20 are also present in a single integrated process column. This construction will be discussed in greater detail below, but it is important to note that this is only a preferred embodiment, and the first and second flash drum 18, 19 and regenerator 20 may be arranged as completely separate services or columns serially connected to each other.

From the first heat exchanger 16, the rich carbonate solution 3 flows throughs second heat exchanger 17. After flowing through the second heat exchanger 17, the temperature of the rich carbonate solution 3 at the second exchanger outlet will have been increased by 5° C. to about 20° C. over the elevated temperature measured at the first exchanger outlet. The heat for the second heat exchanger 17 is supplied from a service outside the scope of this invention.

Using two relatively small and inexpensive heat exchangers rather than a single large and much more expensive exchanger the necessary heat is exchanged while reducing costs. This is especially the case because the rich carbonate solution 3 is pressurized-leaving the bottom of the carbon dioxide absorber at a pressure of up to 20 atm (absolute). Constructing a single heat exchanger to achieve the same amount of heat transfer and thus, the same amount of temperature increase of the rich carbonate solution results in a much larger exchanger, which is considerably more expensive to fabricate. Using two small exchangers also allows one of the exchangers (in FIG. 1, i.e., the first heat exchanger 16) to be sized to be powered exclusively by the sensible heat from the exchanged hot lean solvent stream 11.

From the second heat exchanger 17, the rich carbonate solution 3 flows to the flash drum regenerator 25 and enters the first flash drum section 18. The pressure in the first flash drum 18 is about 2.5 to about 4.5 atm (absolute). Within that range the pressure is regulated and adjusted so that when the rich carbonate solution 3 enters the first flash drum 18 the ethylene in the rich carbonate solution will flash out of the rich carbonate solution and having been so separated, rise upwardly to form the first flash drum overhead 6. Most of the ethylene solubilized in the rich carbonate solution 3 is flashed into the vapor phase to form the first flash drum overhead, specifically at least about 95 wt %, preferably at least about 99 wt % of the ethylene contained in the rich carbonate solution is flashed into the first flash drum overhead 6. The absorbed carbon dioxide and water in the rich carbonate solution 3 have a volatility less than that of ethylene and so they do not enter the vapor phase but instead form a first flash liquid 5 in the bottom of the first flash drum 18. Thus, the rich carbonate solution 3 is divided in the first flash drum 18 into a first flash drum overhead 6 and a first flash liquid 5. As noted above, not all of the ethylene is flashed into the vapor phase—and the remaining 1% to 5% of the ethylene that is not flashed into the first flash drum overheads is contained in the first flash drum liquid 5. This first flash drum liquid 5 flows from the first flash drum 18 by pressure differential to the second flash drum 19—the pressure in the second flash drum 19 is from about 1.5 to about 2.5 atm (absolute). Upon entering the second flash section 19, the first flash liquid 5 is vaporized and form a second flash overhead 7 which is then combined with the first flash overhead 6 and sent to the recovery compressor 22. In the recovery compressor the first and second flash overheads are compressed to the pressure of the ethylene oxide reactor and sent to the reactor. The second flash drum is the last opportunity to remove ethylene and so it is operated to ensure to maximize the amount of ethylene flashed off into the vapor phase and into the second flash overhead 7.

The second flash liquid 8 in the bottom of the second flash drum 19 flows by pressure differential to the regenerator section 20, which operates at near atmospheric pressure. The second flash liquid 8 is an aqueous solution of carbon dioxide and only trace amounts of ethylene.

In the regenerator section 20, the carbon dioxide and trace ethylene oxide are separated from the second flash liquid 8 by steam-stripping to yield a gaseous regenerator overhead stream 9 and a lean solvent 11. The lean solvent 11 is then sent through the first heat exchanger 16 and returned to the carbon dioxide absorber 15 ready to absorb additional carbon dioxide. Since substantially all the ethylene has been removed from the rich carbonate solution by the combination of the heating in the first and second heat exchangers 16 and 17 and the subsequent flashing in flash drums 18 and 19, the amount of ethylene in the overhead steam 9 is less than about 50 ppm, preferably less than about 20 ppm (on weight basis). The overhead stream also contains from about to about 20 wt % to about 35 wt % of carbon dioxide with the balance being mostly water. The overhead stream may thus, be vented to the atmosphere. The regenerator overhead 9 contains a significant amount of excess heat because of the aforementioned steam-stripping that takes place in the regenerator in order to separate the carbon dioxide from the second flash liquid 8. The regenerator overhead 9 stream being the product of a hot liquid stream and steam has a significant amount of sensible heat. If desired this excess heat can be supplied as sensible heat to heat another stream, such as in a heat exchanger, prior to venting the regenerator overhead.

In operation, as noted above, steam is used in the regenerator section to contact the second flash liquid and strip carbon dioxide from the second flash liquid 8. The carbon dioxide (along with trace ethylene) forms the regenerator overhead 9. This steam that is used in the regenerator section is a combination of both live steam imported through line 10 and also reboiler steam from the reboiler 21 that is introduced via line 13. The regenerator section 20 preferably contains column internals positioned at the top of the regenerator to minimize carbonate solution entrainment into the vapor and reduce the carbonate in the regenerator overhead 9. The regenerator section 20 preferably contains additional column internals that promote liquid vapor contact to facilitate the stripping of CO₂ from the second flash liquid 8 by contacting it with steam.

FIG. 1 and FIG. 2 show a preferred construction of the process of the present invention. Particular attention, especially with respect to FIG. 2 showing the flash drum regenerator 25. As can be seen in these figures, the flash drum regenerator 25 is constructed as an integrated process column containing multiple interior service sections to perform a sequence of separation tasks within a single column shell. For the flash drum regenerator 25, the interior is divided into three sections by two vapor and liquid-tight horizontal partitions AA and BB. These partitions are preferably steel plates and as shown in FIG. 2 may have a downwardly arcuate shape. The flash drum regenerator 25 comprises a first flash drum 18, a second flash drum 19, and a regenerator section 20 and is thus, divided into three sections/sections. Partition AA separates the first flash drum 18 and the second flash drum 19, while partition BB separates the second flash drum 19 from the regenerator 20. The feed point for the flash drum regenerator unit is located in the first flash drum—as shown in FIGS. 1 and 2, the rich carbonate solution 4 enters through this feed point. At the other end of the flash drum regenerator is the discharge point through which the lean solvent 11 flows. Between these two points live steam 10 is injected into the regenerator section while the regenerator overhead steam is withdrawn through line 9 as described above. The first flash drum 18 is connected to the second flash drum 19 via a first external liquid line (through which the first flash liquid 5 flows) and the second flash drum 19 is connected to the regenerator section 20 via a second external liquid line (through which the second flash liquid 8 flows). While three sections are shown in FIG. 2, as noted above the integrated process column may contain multiple interior service sections.

### Silver-Based Epoxidation Catalyst

As mentioned above, the present ethylene oxide process makes use of a silver-based epoxidation catalyst. The silver-based epoxidation catalyst includes a support, and at least a catalytically effective amount of silver or a silver-containing compound; also optionally present is a promoting amount of rhenium or a rhenium-containing compound; also optionally present is a promoting amount of one or more alkali metals or alkali-metal-containing compounds. The support employed in this invention may be selected from a large number of solid, refractory supports that may be porous and may provide the preferred pore structure. Alumina is well known to be useful as a catalyst support for the epoxidation of an olefin and is the preferred support.

Regardless of the character of the support used, it is usually shaped into particles, chunks, pieces, pellets, rings, spheres, wagon wheels, cross-partitioned hollow cylinders, and the like, of a size suitable for employment in a fixed-bed epoxidation reactor. The support particles will preferably have equivalent diameters in the range from about 3 mm to about 12 mm, and more preferably in the range from about 5 mm to about 10 mm. (Equivalent diameter is the diameter of a sphere having the same external surface (i.e., neglecting surface within the pores of the particle) to volume ratio as the support particles being employed.) Suitable supports are available from Saint-Gobain Norpro Co., Sud Chemie AG, Noritake Co., CeramTec AG, and Industrie Bitossi S.p.A. Without being limited to the specific compositions and formulations contained therein, further information on support compositions and methods for making supports may be found in U.S. Patent Publication No. 2007/0037991.

In order to produce a catalyst for the oxidation of an olefin to an olefin oxide, a support having the above characteristics is then provided with a catalytically effective amount of silver on its surface. In one embodiment, the catalytic effective amount of silver is from 10% by weight to 45% by weight. The catalyst is prepared by impregnating the support with a silver compound, complex or salt dissolved in a suitable solvent sufficient to cause deposition of a silver-precursor compound onto the support. Preferably, an aqueous silver solution is used.

A promoting amount of a rhenium component, which may be a rhenium-containing compound or a rhenium-containing complex may also be deposited on the support, either prior to, coincidentally with, or subsequent to the deposition of the silver. The rhenium promoter may be present in an amount from about 0.001 wt. % to about 1 wt. %, preferably from about 0.005 wt. % to about 0.5 wt. %, and more preferably from about 0.01 wt. % to about 0.1 wt. % based on the weight of the total catalyst including the support, expressed as the rhenium metal.

Other components which may also be deposited on the support either prior to, coincidentally with, or subsequent to the deposition of the silver and rhenium are promoting amounts of an alkali metal or mixtures of two or more alkali metals, as well as optional promoting amounts of a Group IIA alkaline earth metal component or mixtures of two or more Group IIA alkaline earth metal components, and/or a transition metal component or mixtures of two or more transition metal components, all of which may be in the form of metal ions, metal compounds, metal complexes and/or metal salts dissolved in an appropriate solvent. The support may be impregnated at the same time or in separate steps with the various catalyst promoters. The particular combination of support, silver, alkali metal promoter(s), rhenium component, and optional additional promoter(s) of the instant invention will provide an improvement in one or more catalytic properties over the same combination of silver and support and none, or only one of the promoters.

As used herein the term “promoting amount” of a certain component of the catalyst refers to an amount of that component that works effectively to improve the catalytic performance of the catalyst when compared to a catalyst that does not contain that component. The exact concentrations employed, of course, will depend on, among other factors, the desired silver content, the nature of the support, the viscosity of the liquid, and solubility of the particular compound used to deliver the promoter into the impregnating solution. Examples of catalytic properties include, inter alia, operability (resistance to runaway), selectivity, activity, conversion, stability and yield. It is understood by one skilled in the art that one or more of the individual catalytic properties may be enhanced by the “promoting amount” while other catalytic properties may or may not be enhanced or may even be diminished.

Suitable alkali metal promoters may be selected from lithium, sodium, potassium, rubidium, cesium or combinations thereof, with cesium being preferred, and combinations of cesium with other alkali metals being especially preferred. The amount of alkali metal deposited or present on the support is to be a promoting amount. Preferably, the amount ranges from about 10 ppm to about 3000 ppm, more preferably from about 15 ppm to about 2000 ppm, and even more preferably from about 20 ppm to about 1500 ppm, and as especially preferred from about 50 ppm to about 1000 ppm by weight of the total catalyst, measured as the metal.

Suitable alkaline earth metal promoters comprise elements from Group IIA of the Periodic Table of the Elements, which may be beryllium, magnesium, calcium, strontium, and barium or combinations thereof. Suitable transition metal promoters may comprise elements from Groups IVA, VA, VIA, VIIA and VIIIA of the Periodic Table of the Elements, and combinations thereof.

The amount of alkaline earth metal promoter(s) and/or transition metal promoter(s) deposited on the support is a promoting amount. The transition metal promoter may typically be present in an amount from about 0.1 micromoles per gram to about 10 micromoles per gram, preferably from about 0.2 micromoles per gram to about 5 micromoles per gram.

The silver solution used to impregnate the support may also comprise an optional solvent or a complexing/solubilizing agent such as are known in the art. A wide variety of solvents or complexing/solubilizing agents may be employed to solubilize silver to the desired concentration in the impregnating medium. Useful complexing/solubilizing agents include amines, ammonia, oxalic acid, lactic acid and combinations thereof. Amines include an alkylene diamine having from 1 to 5 carbon atoms. In one preferred embodiment, the solution comprises an aqueous solution of silver oxalate and ethylene diamine. The complexing/solubilizing agent may be present in the impregnating solution in an amount from about 0.1 to about 5.0 moles per mole of silver, preferably from about 0.2 to about 4.0 moles, and more preferably from about 0.3 to about 3.0 moles for each mole of silver.

When a solvent is used, it may be an organic solvent or water, and may be polar or substantially or totally non-polar. In general, the solvent should have sufficient solvating power to solubilize the solution components. At the same time, it is preferred that the solvent be chosen to avoid having an undue influence on or interaction with the solvated promoters. Organic-based solvents which have 1 to about 8 carbon atoms per molecule are preferred. Mixtures of several organic solvents or mixtures of organic solvent(s) with water may be used, provided that such mixed solvents function as desired herein.

The concentration of silver in the impregnating solution is typically in the range from about 0.1% by weight up to the maximum solubility afforded by the particular solvent/solubilizing agent combination employed. It is generally very suitable to employ solutions containing from 0.5% to about 45% by weight of silver, with concentrations from 5 to 35% by weight of silver being preferred.

Impregnation of the selected support is achieved using any of the conventional methods; for example, excess solution impregnation, incipient wetness impregnation, spray coating, etc. Typically, the support material is placed in contact with the silver-containing solution until a sufficient amount of the solution is absorbed by the support. Preferably the quantity of the silver-containing solution used to impregnate the porous support is no more than is necessary to fill the pores of the support. A single impregnation or a series of impregnations, with or without intermediate drying, may be used, depending, in part, on the concentration of the silver component in the solution. Impregnation procedures are described, for example, in U.S. Pat. Nos. 4,761,394, 4,766,105, 4,908,343, 5,057,481, 5,187,140, 5,102,848, 5,011,807, 5,099,041 and 5,407,888. Known prior procedures of pre-deposition, co-deposition and post-deposition of various the promoters can be employed.

After impregnation of the support with the silver-containing compound, i.e., a silver precursor, a rhenium component, an alkali metal component, and the optional other promoters, the impregnated support is calcined for a time sufficient to convert the silver containing compound to an active silver species and to remove the volatile components from the impregnated support to result in a catalyst precursor. The calcination may be accomplished by heating the impregnated support, preferably at a gradual rate, to a temperature in the range from about 200° C. to about 600° C. at a pressure in the range from about 0.5 to about 35 bar. In general, the higher the temperature, the shorter the required heating period. A wide range of heating periods have been suggested in the art; e.g., U.S. Pat. No. 3,563,914 discloses heating for less than 300 seconds, and U.S. Pat. No. 3,702,259 discloses heating from 2 to 8 hours at a temperature of from 100° C. to 375° C., usually for duration of from about 0.5 to about 8 hours. However, it is only important that the heating time be correlated with the temperature such that substantially all of the contained silver is converted to the active silver species. Continuous or step-wise heating may be used for this purpose.

During calcination, the impregnated support may be exposed to a gas atmosphere comprising an inert gas or a mixture of an inert gas with from about 10 ppm to 21% by volume of an oxygen-containing oxidizing component. For purposes of this invention, an inert gas is defined as a gas that does not substantially react with the catalyst or catalyst precursor under the conditions chosen for the calcination. Further information on catalyst manufacture may be found in the aforementioned U.S. Patent Publication No. 2007/0037991.

### EXAMPLE

The invention will now be described in more detail with respect to the following non-limiting examples.

A 80.6 KTA ethylene-oxide equivalent capacity plant was simulated using PRO/II software. For these simulations, the selectivity was set to 85% and the carbon dioxide reactor inlet concentration was 0.5%. The operation of the carbon dioxide absorber, preheater and regenerator were then simulated under three different cases as set forth below in Table 1. The reference numbers in Table 1 refer to the streams and services. Case I is the prior art, where no second heat exchanger is used. Cases II and III are according to the present invention, with the use of an additional preheater #17. In Case II, preheater #17 increases the temperature of the rich carbonate solution to 110° C., while in Case III preheater #17 increases the temperature of the rich carbonate solution to 115° C. The performance improvement resulting from the Cases II and III is shown in the last two columns of Table 1, below; and is further broken out in Table 2.

TABLE 1

| | Case No. I | Case No. II | Case No. III |
|---|---|---|---|
| | (prior Art) | (invent.) | (invent.) |
| #3 Absorb. Bottoms Liquid | 66 | 66 | 66 |
| Temperature | | | |
| (° C.) | | | |
| #3 | 20.2 | 20.2 | 20.2 |
| Absorb. Bottoms Liquid | | | |
| pressure | | | |
| (kg/cm²) | | | |
| #3 | 9572 | 9572 | 9572 |
| Absorb. Bottoms Liquid | | | |
| Flow Rate | | | |
| (kmol/hr) | | | |
| #3 | 72 | 72 | 72 |
| [ethylene] | | | |
| in Absorb. Bottoms Liquid | | | |
| (kg/hr) | | | |
| #16 | 105 | 105 | 105 |
| First | | | |
| Exchanger | | | |
| outlet | | | |
| temperature | | | |
| (“elevated temperature”) | | | |
| (° C.) | | | |
| #17 | — | 110 | 115 |
| Second | | | |
| Exchanger outlet | | | |
| temperature | | | |
| (° C.) | | | |
| #18 | 2.5 | 2.5 | 2.5 |
| First Flash | | | |
| Drum Pressure | | | |
| (kg/cm²) | | | |
| #6 | 339 | 449 | 695 |
| First | | | |
| Flash vapor overhead | | | |
| Flow Rate | | | |
| (kg/hr) | | | |
| #6 | 69 | 70 | 71 |
| [ethylene] | | | |
| in First Flash vapor Overhead | | | |
| (kg/hr) | | | |
| #6 | 96 | 146 | 261 |
| [CO₂] | | | |
| in First Flash | | | |
| Vapor Overhead | | | |
| (kg/hr) | | | |
| #5 | 104 | 106 | 114 |
| First | | | |
| Flash liquid bottoms | | | |
| (° C.) | | | |
| #5 | 3.1 | 2.3 | 1.5 |
| [ethylene] | | | |
| In First Flash liquid bottoms | | | |
| (kg/hr) | | | |
| #19 | 1.58 | 1.58 | 1.58 |
| Second | | | |
| Flash | | | |
| Drum | | | |
| pressure | | | |
| (kg/cm²) | | | |
| #7 | 13 | 559 | 688 |
| Second Flash Vapor overhead | | | |
| Flow Rate | | | |
| (kg/hr) | | | |
| #7 | 2 | 2.26 | 1.5 |
| Second | | | |
| Flash vapor | | | |
| Overhead | | | |
| [ethylene] (kg/hr) | | | |
| #7 | 30 | 248 | 1763 |
| Second | | | |
| Flash vapor | | | |
| Overhead | | | |
| [CO₂] (kg/hr) | | | |
| #8 | 0.8 | 0.04 | 0.01 |
| Second Flash liquid bottoms | | | |
| [ethylene] | | | |
| (kg/hr) | | | |
| #9 | 3550 | 3550 | 3550 |
| Regen. | | | |
| Vapor | | | |
| overhead | | | |
| [CO₂] | | | |
| (kg/hr) | | | |
| #9 | 0.8 (274 | 0.04 (14 | 0.01(3 |
| Regen. | mol ppm) | mol ppm) | mol ppm) |
| Vapor | | | |
| overhead | | | |
| [ethylene] | | | |
| (kg/hr) | | | |
| Reduction in ethylene vented | — | 20x | 93x |
| to Atmosphere relative to Case I | | | |

TABLE 2

| | | #16 | | | |
|---|---|---|---|---|---|
| | | First | | | |
| | #9 | Exchanger | #17 | #9 | |
| | Regen. | outlet | Second | Regen. | |
| | Vapor | temperature | Exchanger | Vapor | Reduction in |
| | Overhead | (“elevated | outlet | Overhead | ethylene vented to |
| | [ethylene] | temperature”) | temperature | [ethylene] | Atmosphere relative |
| Case No. | (kg/hr) | (° C.) | (° C.) | (mol ppm) | to Case I |
| I (prior Art) | 0.8 | 105 | — | (274 mol | — |
| | | | | ppm) | |
| II (invention) | 0.04 | 105 | 110 | (14 mol | 20x |
| | | | | ppm) | |
| III (invention) | 0.01 | 105 | 115 | (3 mol | 93x |
| | | | | ppm) | |

Table 2 compares the reduction in ethylene vented to the atmosphere across three cases. In Case I the process is configured and operated according to the prior art (no preheater) while in Cases II and III the process is operated according to the present invention by insertion of an additional preheater #17. Further in Case II the preheater #17 increases the temperature of the rich carbonate solution from the Case I temperature of 105° C. to 110° C., while in Case III the preheater #17 increases the temperature of the rich carbonate solution to 115° C.

As shown above, when the process is configured and operated according to the present invention, the amount of ethylene vented to the atmosphere is significantly reduced compared to prior art operation—by 20× when the preheater increases the temperature of the rich carbonate solution to 110° C. and by 93× when the temperature is increased to 115° C.

While the present disclosure has been particularly shown and described with respect to preferred embodiments thereof, it will be understood by those skilled in the art that the foregoing and other changes in forms and details may be made without departing from the spirit and scope of the present disclosure. It is therefore intended that the present disclosure not be limited to the exact forms and details described and illustrated, but fall within the scope of the appended claims.

We claim:

1. A process for preparation of ethylene oxide comprising the steps of:
(a) providing scrubber overheads comprising about 25 vol % to about 35 vol % ethylene;
(b) in a carbon dioxide absorber, contacting the scrubber overheads with a carbon dioxide-absorbing solvent to form a remaining gas stream and a rich carbonate solution, the rich carbonate solution comprising ethylene having a temperature of about 65° C. to about 85° C.;
(c) passing the rich carbonate solution under a pressure of between about 10 to about 20 atm to and through a first heat exchanger, where the temperature of the rich carbonate solution is raised to an elevated temperature after exiting the first heat exchanger;
(d) directing the rich carbonate solution from the first heat exchanger to and through a second heat exchanger, where the temperature of the rich carbonate solution is raised to about 5° C. to about 20° C. above the elevated temperature;
(e) dividing, in a first flash drum, the rich carbonate solution into a first flash drum overhead and a first flash liquid;
(f) separating, in a second flash drum, a second flash liquid from the first flash liquid;
(g) passing the second flash liquid to a regenerator; and
(h) dividing, in the regenerator, the second flash liquid into a lean solvent and a gaseous regenerator overhead steam comprising carbon dioxide and ethylene, wherein ethylene is present in an amount of less than about 50 ppm (on weight basis).

2. The process of claim 1, wherein the rich carbonate solution contains from about 0.01 to about 0.5 wt % of ethylene.

3. The process of claim 1, the elevated temperature being about 95° C. to about 110° C.

4. The process of claim 1, wherein, in the first flash drum, at least 95% of the ethylene in the rich carbonate solution is flashed into the first flash drum overhead.

5. The process of claim 1, wherein, in the first flash drum, at least 95% of the ethylene in the rich carbonate solution is flashed into the first flash drum overhead and the pressure in the first flash drum is about 2.5 to about 4.5 atm (absolute).

6. The process of claim 1, wherein in step (b), substantially all of the scrubber overheads are sent to the carbon dioxide absorber and contacted with a carbon dioxide-absorbing solvent.

7. The process of claim 1, wherein the gaseous regenerator overhead stream comprises from about 20 wt % to about 35 wt % carbon dioxide.

8. The process of claim 1, further including an integrated process column flash drum regenerator, wherein the integrated process column flash drum regenerator comprises the first flash drum, the second flash drum, and the regenerator.

9. The process of claim 8, wherein the first flash drum is separated from the second flash drum by a vapor and liquid-tight horizontal partition, and the second flash drum is separated from the regenerator by a second vapor and liquid-tight horizontal partition.

10. The process of claim 8, wherein the first flash drum is separated from the second flash drum by a vapor and liquid-tight horizontal partition, and the second flash drum is separated from the regenerator by a second vapor and liquid-tight horizontal partition and the first flash drum is connected to the second flash drum by an external liquid line, and the second flash drum is connected to the regenerator by a second external liquid line.

11. The process of claim 1, wherein the amount of ethylene is less than about 20 ppm (on weight basis).

12. The process of claim 1, wherein, in the first flash drum, at least 99% of the ethylene in the rich carbonate solution is flashed into the first flash drum overhead.
